(19) United States
(12) Patent Application Publication
(10) Pub. No.: US 2017/0265958 A1
(43) Pub. Date: Sep. 21, 2017

(54) DIABETES TESTING KIT AND METHODS FOR ITS USE

(71) Applicant: Medical Monofilament Manufacturing LLC, Plymouth, MA (US)

(72) Inventors: Robert Fox, Mashpee, MA (US); Thomas J. Hardiman, Plymouth, MA (US); Michelle Hardiman, Plymouth, MA (US)

(21) Appl. No.: 15/071,323

(22) Filed: Mar. 16, 2016

(51) Int. Cl. A61B 50/30; A61B 5/00

(52) U.S. Cl. CPC A61B 50/30; A61B 5/0048; A61B 5/0079; A61B 5/6841; A61B 5/4041; A61B 5/4827; A61B 2050/0067

(57) ABSTRACT

Methods and apparatuses for use in screening for and assessing neuropathies, such as severe diabetic neuropathies of the feet, also known as Loss of Protective Sensation (“LOPS”). The methods and apparatuses described herein employ a kit including one or more of an insert having at least one testing instrument attached thereto, the at least one testing instrument comprising at least a holder and a monofilament; an enclosure including inner compartments for holding and securing the insert; a folder having one or more pockets for securing the insert and one or more of a mirror, a packet and other material, such as instructions. The apparatuses safely secure and protect the testing instrument(s) so that the risk of breaking or otherwise damaging the testing instruments during storage and/or shipping is significantly reduced. The methods and apparatuses are easy and low cost to manufacture, easy to use, scalable, and allow users to self-screen/test for, among other things, severe diabetic neuropathies of the feet.

### BACKGROUND

Technical Field

This application relates generally to apparatuses and methods of using the apparatuses for testing/assessing tactile sensitivity associated with neuropathies, and, more particularly to diabetes testing kits and methods of their use for assessing diabetic neuropathies and preventing lower extremity amputations.

Background Information

It is estimated that more than 29 million people in the United States are afflicted with diabetes. Diabetes is a very serious disease, complications from which may lead to disabling conditions such as blindness, nerve damage and/or amputation, or to potentially fatal problems such as heart disease, stroke and/or kidney failure. Currently, diabetes is the seventh leading cause of death among Americans.

Approximately 15-20% of the diabetic population will develop foot (i.e. plantar) ulcers during their lifetime due to diabetic neuropathy. In people with diabetes, 85% of all lower extremity amputations are preceded by a foot ulceration that has resulted in severe infection. Moreover, approximately 50% of these amputees develop foot ulcers on their non-affected foot within eighteen months of amputation. This affliction affects the bottoms (i.e. plantar aspect) of the feet, and manifests itself via a gradual loss of sensation that begins in the toes and ultimately progresses back toward the heel.

Worldwide, a lower extremity amputation is performed every 20 seconds due to diabetes. Furthermore, although 60-70% of people with diabetes will develop neuropathy, most of these people are unaware of the condition. People with severe neuropathy, also known as Loss of Protective Sensation (“LOPS”), are 7 times more likely to develop a foot ulceration.

To screen for loss of foot sensation among diabetics, a clinician tests each foot of a patient by pressing a testing implement against each predetermined location until the testing device buckles, all-the-while monitoring whether the patient can detect the force created by the testing implement against his/her feet. A patient's inability to detect sensation will signal to the tester that the patient is at high risk for plantar ulceration. Certain testing devices for detecting sensation are described in U.S. Pat. No. 6,790,304.

However, existing technologies for assessing neuropathies, such as diabetic neuropathies, often require traveling to a physician's office to have medical personnel conduct various tests. Existing technologies are also disjointed as various testing instruments and recording media are haphazardly used and assembled at a first and subsequent patient visit(s). Moreover, existing technologies do not provide methods and apparatuses capable of effectively securing and/or holding the required testing instruments, recording media and/or instructions, so as to decrease the risk of breaking or distorting (such as bending and the like) of the testing instruments during storage and/or shipping thereof. The methods and apparatuses discussed in the illustrative embodiments below solve these problems and also offer additional advantages not offered by current or past testing procedures and apparatuses.

### SUMMARY

Methods and apparatuses described herein employ a kit including one or more of: an insert, having at least one testing instrument attached thereto, the at least one testing instrument comprising at least a holder and a monofilament; an enclosure including inner compartments for holding and securing the insert; and a folder having one or more pockets for securing the insert and one or more of a mirror, a packet and/or other material, such as instructions. The methods and apparatuses may be used to assess/screen-for neuropathy, such as diabetic neuropathy, including severe diabetic neuropathy. The methods and apparatuses are easy and low cost to manufacture, easy to use and scalable. The methods and apparatuses also provide a user with complete, low cost, reliable and easy means to organize materials necessary to self-screen and/or assess diabetic neuropathy, thus making the overall prevention of diabetic foot complications much more likely.

Moreover, the apparatuses safely secure and protect the testing instrument(s) so that the risk of breaking, distorting (such as bending and the like) or otherwise damaging the testing instruments during storage and/or shipping is significantly reduced. The methods and apparatuses allow users to self-screen and/or assess, among other things, tactile sensitivity, or loss thereof, associated with diabetic neuropathy, including severe diabetic neuropathy, in the comfort of their own homes.

### BRIEF DESCRIPTION OF THE DRAWINGS

The embodiments described below refer to the accompanying drawings, of which:

FIG. 1 is an elevational view of an insert of an illustrative embodiment;

FIG. 2 is a perspective view of an enclosure of an illustrative embodiment;

FIG. 3 is a perspective view of a folder of an illustrative embodiment;

FIG. 4 is a perspective view of a folder of an illustrative embodiment;

FIGS. 5A-D are perspective views of inserts of illustrative embodiments;

FIGS. 6A-B are perspective views of an insert and a packet of an illustrative embodiment;

FIGS. 7A-E are perspective views of a mirror and a packet of an illustrative embodiment;

FIG. 8 is a perspective view of a folder and a packet of an illustrative embodiment; and

FIGS. 9A-E is an overview of a method of an illustrative embodiment.

### DETAILED DESCRIPTION OF AN ILLUSTRATIVE EMBODIMENT

Diabetes testing kits and methods for their use are discussed in more detail below. Such methods and apparatuses are merely illustrative and should not be considered as limiting.

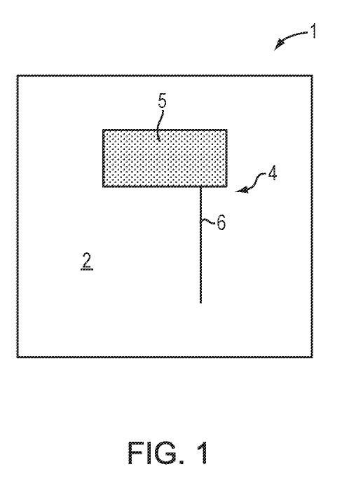

As illustrated in FIG. 1, a square insert 1 of a testing kit includes a front face 2. Securely attached to the front face 2 is a testing instrument 4 having a holder 5 and a monofilament 6. The monofilament 6 may be the type described in U.S. Pat. No. 6,790,304, such as, for example, a 10 gram monofilament made of nylon or other polymers. The example monofilament 6 has a distal end and a proximal end, the proximal end attached to the holder 5. In this illustrative embodiment, the testing instrument 4 is attached to the front face 2 by glue. In other embodiments, the testing instruments may be attached to a front face of an insert by any attachment means capable of maintain the testing instrument securely on the insert so as to allow a user to remove the testing instrument without damaging or otherwise distorting either the insert or the testing instrument, such means includes, but is not limited to, clip-on(s), Velcro®, paperclip(s), other adhesives (e.g. other glue-like adhesives, such as cement, mucilage, paste, or the like) and/or adhesive material (e.g. tape).

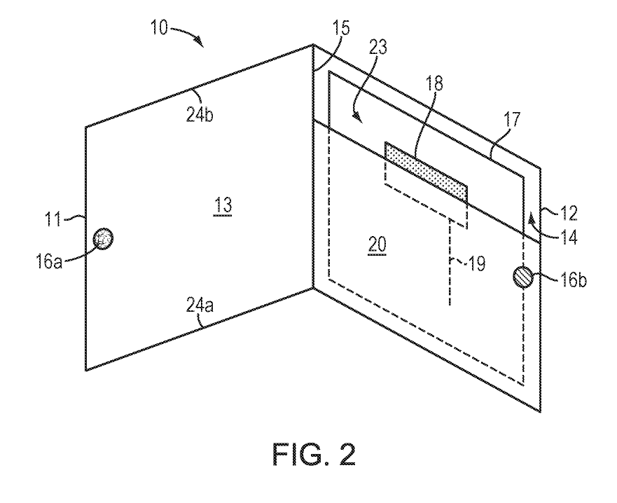

In the illustrative embodiment of FIG. 2, a diabetes testing kit includes an enclosure 10 having an open position and a closed position (not shown). The enclosure 10 includes a front panel 11 and a rear panel 12 joined by a spine 15. The front panel 11 has a back face 13. The back panel 12 has a front face 14. The back face 13 has a bottom side 24a and a top side 24b. The enclosure 10 has a vertical axis extending along the back face 13 and perpendicularly from the bottom side 24a to the top side 24b, or vice versa. The enclosure 10 also has a longitudinal axis extending along the back face 13 from the edge of front panel 11 to the spine 15 and parallel to the bottom side 24a and the top side 24b. The vertical and longitudinal axes are similarly positioned on the rear panel 12.

The rear panel 12 includes a front face 14 and an inner compartment 20 on the front face 14. The inner compartment 20 extends along the longitudinal axis across the front face 14 at about a quarter (¼) way down the vertical axis along the rear panel 12. The inner compartment 20 holds an insert 17 securely therein. In the illustrative embodiment of FIG. 2, the insert 17 includes at least one testing instrument 23 having a holder 18 and a monofilament 19. The inner compartment 20 is of sufficient depth to secure the testing instrument 23 at the holder 18 so as to, among other things, safeguard the monofilament 19 from damage, including breaking, distorting or deforming. In other embodiments, the inner compartment 20 may be located at different positions and heights on the face 14 along the vertical axis of the rear panel 12.

The enclosure 10 may also have one or more securements to secure the enclosure in the closed position. In the illustrative embodiment of FIG. 2, the enclosure 10 includes Velcro® securements 16a and 16b. The securement 16a is located on the edge of the back face 13 at about a halfway point on the vertical axis of the front panel 11. The securement 16b corresponds to the position of the securement 16a and is located on the inner compartment 20 at about a halfway point along the vertical axis of the rear panel 12. As the enclosure 10 closes, such as in FIG. 2, the Velcro® of securements 16a and 16b catch to maintain the enclosure 10 in the closed position while also providing easy opening of the enclosure 10. Alternatively, the enclosure 10 may have an open position and a closed position, but may not include securements 16a and 16b, such that the closed position is maintained by, among other things, the strength of a crease of spine 15.

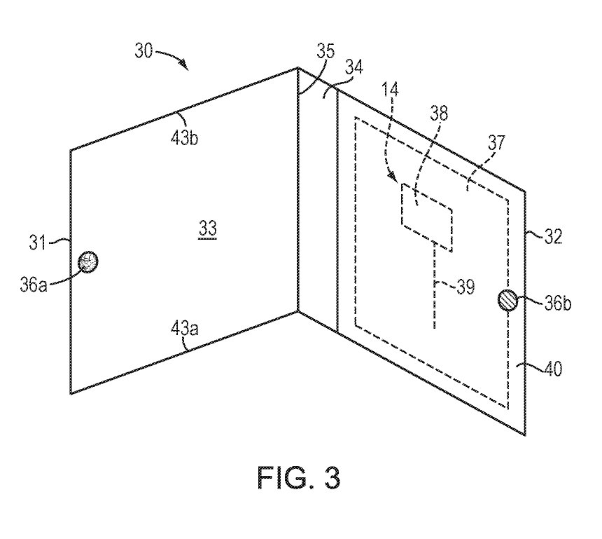

Referring now to FIG. 3, a diabetes testing kit comprises a folder 30 having an open position and a closed position (not shown). The folder 30 includes a front panel 31 and a rear panel 32 joined by a spine 35. The front panel 31 has a back face 33 and the rear panel 32 has a front face 34. The back face 33 has a bottom side 43a and a top side 43b. The folder 30 has a vertical axis extending along the back face 33 and perpendicularly from the bottom side 43a to the top side 43b, or vice versa. The folder 30 also has a longitudinal axis extending along the back face 43 from the edge of front panel 31 to the spine 35 and parallel to the bottom side 43a and the top side 43b. The vertical and longitudinal axes are similarly positioned on the rear panel 32.

The rear panel 32 includes a front face 34 and a pocket 40 on the front face 34. The pocket 40 extends up the vertical axis across the front face 34 parallel to the spine 35 at about a quarter (¼) way along the longitudinal axis of the rear panel 32 as the rear panel extends outward from the spine 35. The pocket 40 holds an insert 37 securely therein. In this illustrative embodiment, the insert 37 includes at least one testing instrument 44 having a holder 38 and a monofilament 39. The pocket 40 is of sufficient placement and size to secure the testing instrument 44 inside so as to, among other things, safeguard the monofilament 39 and prevent it from damage, including breaking, distorting or deforming. In other embodiments, the pocket 40 may be larger or smaller on the face 34.

The folder 30 may also have one or more securements to secure the folder in the closed position. In the illustrative embodiment of FIG. 3, the folder 30 includes Velcro® securements 36a and 36b. The securement 36a is located on the edge of the back face 33 at about a halfway point on the vertical axis of the front panel 31. The securement 36b corresponds to the position of the securement 36a and is located on the pocket 40 at about a halfway point along the vertical axis of the rear panel 32. As the folder 10 closes, such as in FIG. 3, the Velcro® of securements 36a and 36b catch to maintain the folder 30 in the closed position while also providing easy opening of the folder 30.

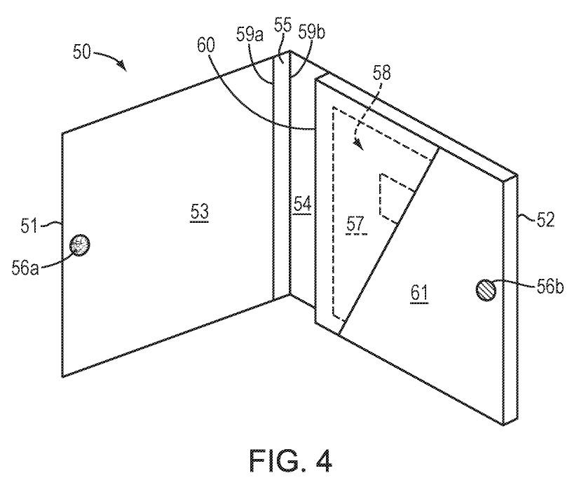

FIG. 4 depicts an alternative embodiment of the diabetes testing kit having at least two inner pockets and a double-creased spine. The diabetes testing kit is comprised of a folder 50 having an open position and a closed position (not shown). The folder 50 includes a front panel 51 and a rear panel 52 joined by a spine 55. The spine 55 includes two creases 59a and 59b.

The front panel 51 has a back face 53 and the rear panel 52 has a front face 54. The rear panel 52 includes a front face 54 and a first inner pocket 60. Similar to the alternative illustrative embodiment shown in FIG. 3, the folder 50 depicted in the illustrative embodiment of FIG. 4 has a vertical axis and a longitudinal axis, the spine 55 running along the vertical axis. The first inner pocket 60 extends up the vertical axis across the front face 54 parallel to the spine 55 at about a quarter (¼) way along the longitudinal axis of the rear panel 52.

The first inner pocket 60 holds an insert 57 securely therein. In this illustrative embodiment, the insert 57 includes at least one testing instrument 58. The first inner pocket 60 is of sufficient placement and size to secure the testing instrument 58 inside the first inner pocket 60 so as to, among other things, prevent the testing instrument 58 from damage, including breaking, distorting or deforming, and/or the insert 57 from falling out of the first inner pocket 60. In other embodiments, the first inner pocket 60 may be larger or smaller on the face 54.

The folder 50 also includes a second inner pocket 61 located on top of the first inner pocket 60 on the rear panel 52. The second inner pocket may be of equal or lesser size compared to the first inner pocket 60 and/or may be angled differently from the first inner pocket 60. In the illustrative embodiment depicted in FIG. 4, the second inner pocket 61 begins and/or opens at about a quarter (¼) of the length of the first inner pocket (along the longitudinal axis) and extends along the vertical plane at an angle so as to connect with the opposite side of the second inner pocket 60 at about a middle point of a side (not labeled) of the first inner pocket 60. The second inner pocket 61 is configured to hold various other inserts (not shown).

The folder 50 may also have one or more securements to secure the folder 50 in the closed position. In the illustrative embodiment of FIG. 4, the folder 50 includes Velcro® securements 56a and 56b. The securement 56a is located on the edge of the back face 53 at about a halfway point on the vertical axis of the front panel 51. The securement 56b corresponds to the position of the securement 56a and is located on the second inner pocket 61 at about a halfway point along the vertical axis of the rear panel 52. As the folder 50 closes, the Velcro® of securements 56a and 56b attach together to maintain the folder 50 in the closed position while also providing easy opening of the folder 50.

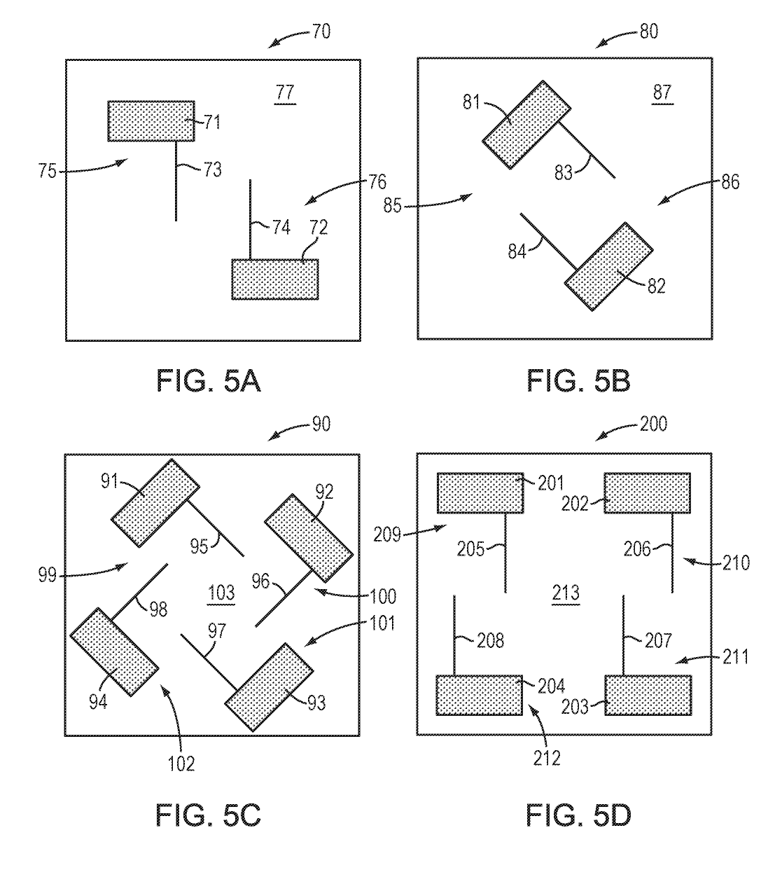

Referring now to FIGS. 5A-D, a square insert 70 has a front face 77 and a back face (not shown) each having four corners. The front face 77 includes a first testing instrument 75 having a holder 71 and a monofilament 73. The front face 77 also includes a second testing instrument 76 having a holder 72 and a monofilament 74. The first testing instrument 75 is located at about two edges of one corner of the front face 77 and positioned so as to have four sides of the holder 71 about parallel to four sides of the insert 70 and an about 90° angle of the holder 71 corresponding to a 90° angle in the respective corner of the insert 70. The monofilament 73 is positioned centrally on the face 77 so as to be furthest away from all four sides of the insert 70.

Similarly, the second testing instrument 76 is located at about two edges of an opposite corner of the front face 77 (i.e. opposite from the corner on which the testing instrument 75 is located) and positioned so as to have four sides of the holder 72 about parallel to four sides of the insert 70 and an about 90° angle of the holder 72 corresponding to a 90° angle in the respective corner of the insert 70. The monofilament 74 is positioned centrally on the face 77 so as to be furthest away from all four sides of the insert 70. The positioning of the monofilaments 73 and 74 on the face 77, for example as depicted in FIG. 5A, helps protect these monofilaments from damage, including breaking, distorting or deforming, during shipping, storage, etc.

In an alternative illustrative embodiment depicted in FIG. 5B, a square insert 80 has a front face 87 and a back face (not shown) each having four corners. The front face 87 includes a first testing instrument 85 having a holder 81 and a monofilament 83. The front face 87 also includes a second testing instrument 86 having a holder 82 and a monofilament 84. Compared to the illustrative embodiment depicted in FIG. 5A, the testing instruments 85 and 86 are skewed by about 45° relative to the positions of the testing instruments 75 and 76 on the insert 70 in FIG. 5A. More specifically, the first testing instrument 85 is located close to two edges of one corner of the front face 87 and positioned so as to have four sides of the holder 81 each at about a 45° angle with respect to each of the four sides of the insert 80.

Similarly, the second testing instrument 86 is located close to two edges of an opposite corner of the front face 87 (i.e. opposite from the corner on which the testing instrument 85 is located) and positioned so as to have four sides of the holder 82 each at about a 45° angle with respect to each of the four sides of the insert 80. The monofilaments 83 and 84 are positioned centrally on the face 87 so as to be furthest away from all four sides of the insert 80 while also being slightly separated from each other on the face 87. The positioning of the monofilaments 83 and 84 on the face 87, for example as depicted in FIG. 5B, helps protect these monofilaments from damage, including breaking, distorting or deforming, during shipping, storage, etc.

In FIG. 5C, a square insert 90 has a front face 103 and a back face (not shown), the front face 103 including testing instruments 99, 100, 101 and 102, each located at one of four corners of the front face 103. In a first corner (not labeled) of the front face 103, the testing instrument 99 is located close to two edges of the first corner and positioned so as have four sides of a holder 91 each at about a 45° angle with respect to each of the four sides of the insert 90. In second corner (not labeled) of the front face 103, the testing instrument 100 is located close to two edges of the second corner and positioned so as to have four sides of a holder 92 each at about a 45° angle with respect to each of the four sides of the insert 90. In a third corner (not labeled) of the front face 103 that is opposite the first corner, the testing instrument 101 is located close to two edges of the third corner and positioned so as to have four sides of a holder 93 each at about a 45° angle with respect to each of the four sides of the insert 90.

In a fourth corner (not labeled) of the front face 103 that is opposite the second corner, the testing instrument 102 is located close to two edges of the fourth corner and positioned so as to have four sides of a holder 94 each at about a 45° angle with respect to each of the four sides of the insert 90. Each of monofilaments 95, 96, 97 and 98 are positioned so as to be located farthest away from all of the four sides of the insert 90 while also being slightly separated from each other. This positioning of these monofilaments helps protect these monofilaments from damage, including breaking, distorting or deforming, during shipping, storage, etc.

As shown in another illustrative embodiment depicted in FIG. 5D, a square insert 200 has a front face 213 having testing instruments 209, 210, 211 and 212 located at each of one of four corners of the insert 200. In a first corner (not labeled) of the front face 213, the testing instrument 209 is located on two edges of the first corner and positioned so as to have each of four sides of a holder 201 about parallel to four sides of the insert 200 and an about 90° angle of the holder 201 corresponding to a 90° angle in the first corner of the insert 200. In a second corner (not labeled) of the front face 213, the testing instrument 210 is located essentially on two edges of the second corner and positioned so as to have each of four sides of a holder 202 about parallel to four sides of the insert 200 and an about 90° angle of the holder 202 corresponding to a 90° angle in the second corner of the insert 200.

In a third corner (not labeled) of the front face 213 that is opposite the first corner, the testing instrument 211 is located essentially on two edges of the third corner and positioned so as to have each of four sides of a holder 203 about parallel to four sides of the insert 200 and an about 90° angle of the holder 203 corresponding to a 90° angle in the third corner of the insert 200. In a fourth corner (not labeled) of the front face 213 that is opposite the first corner, the testing instrument 212 is located essentially on two edges of the fourth corner and positioned so as to have each of four sides of a holder 204 about parallel to four sides of the insert 200 and an about 90° angle of the holder 203 corresponding to a 90° angle in the fourth corner of the insert 200.

In FIG. 5D, each of the monofilaments 205, 206, 207 and 208 are located off center from a vertical axis of their respective holders and attached to their respective holders at about equal distances. One group of the testing instruments that is opposite each other, testing instruments 209 and 211, has monofilaments 205 and 207 positioned centrally on the face 213 so as to be farthest away from all four sides of the insert 200. A second group of testing instruments that is opposite each other, testing instruments 210 and 212, has monofilaments 207 and 208 protruding centrally away from their respective holders 203 and 204 so as to run parallel to two sides of the insert 200 and closer to two sides of the insert 200 than the monofilaments 205 and 206. This positioning of these monofilaments helps protect these monofilaments from damage, including breaking, distorting and/or deforming, during shipping, storage, etc.

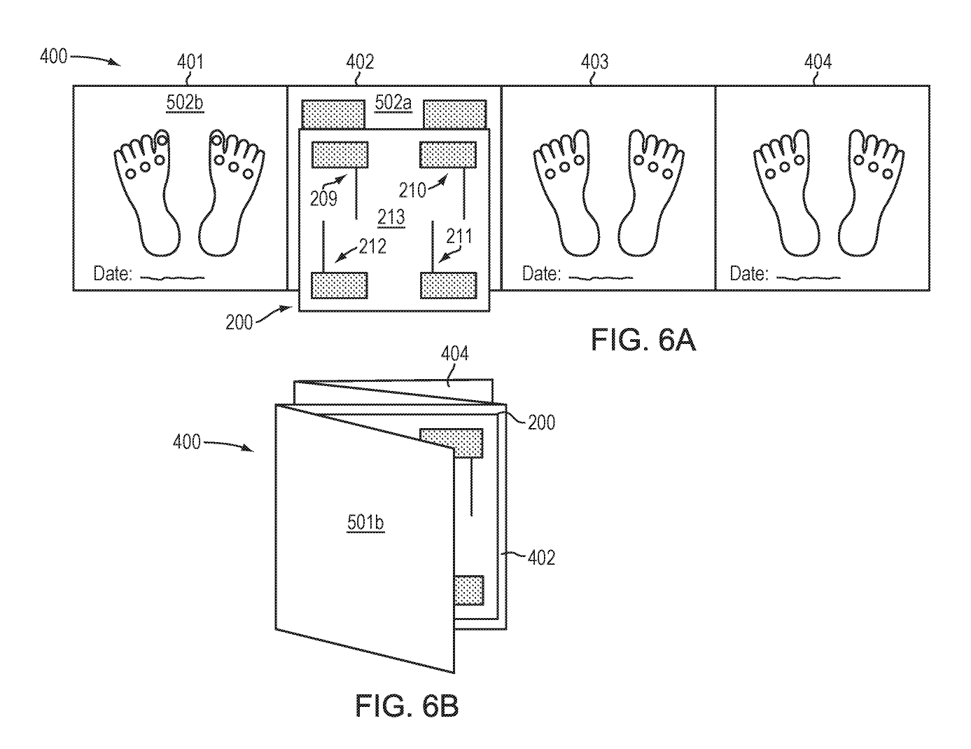

The diabetes testing kit may also include a packet having one or more markable sheets for recording the results of one or more spots of testing of any of the aforementioned testing instruments. In the illustrative embodiment of FIGS. 6A-B, a packet 400 is comprised of four, square, joined markable sheets 401, 402, 403 and 404. These markable sheets may be marked as seen fit by a user, such as, for example, marking at least four areas of a right and left foot corresponding to the testing areas of any of the aforementioned testing instruments and marking the date of testing. The packet 400 is specially configured so as to fold in an accordion-like manner as shown, for example, in FIG. 6B. The markable sheets have front faces and back faces, such as, for example, the markable sheet 401 having a front face 501A and a back face 501B and the markable sheet 402 having a front face 502A. The markable sheets 401, 402, 403 and 404 may be made of paper or any medium capable of recording results.

FIG. 6A depicts an illustrative embodiment wherein the insert 200 of FIG. 5D is placed inside the packet 400 over the front fact 502A. As shown in FIG. 6B, when the packet 400 is folded in the accordion-like manner, the insert 200 (and the testing instruments 209, 210, 212 and 211) is secured therein, and the testing instruments are protected from damage, including breaking, distorting and/or deforming, by markable sheet 401 folding over markable sheet 402.

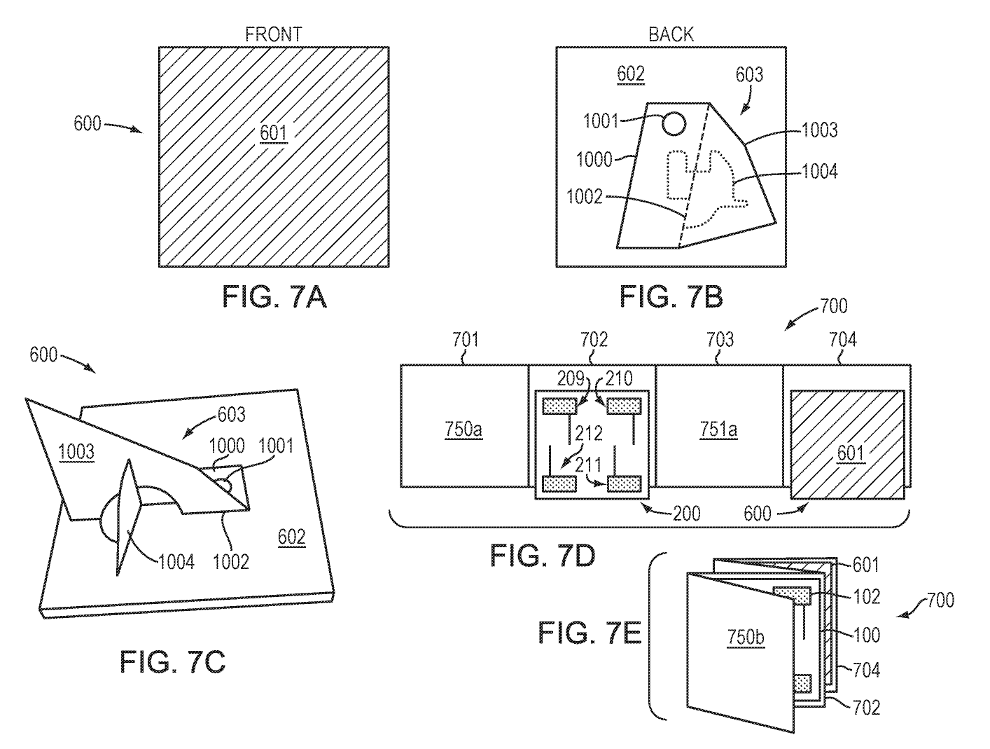

The testing kits discussed herein may also include a mirror 600 as shown in the illustrative embodiments of FIGS. 7A-E. FIGS. 7A-B depict the mirror 600 having a front face 601 with a reflective surface and a back face 602 without a reflective surface. The mirror 600 also includes a foldable stand 603 attached to the back face 602. The foldable stand 603 includes a main portion 1000 with a hole 1001 and a wing 1003 having a separable portion 1004 extending from the wing 1003 to the main portion 1000. The main portion 1000 and the wing 1003 are divided by a fold 1002.

When using the foldable stand 603, a user bends the wing 1003 away from the face 602 and along the fold 1002. The user then separates the separable portion 1004 from the wing 1003 and turns the separable portion 1004 so as to lock the wing portion 1003 in its folded-out position as seen in FIG. 7C. The foldable stand 603 thus allows the user to use the mirror 600 without hands by placing the mirror 600 on a surface.

FIG. 7D illustrates the mirror 600 having the additional functionality of being collapsible and of sufficient size to fit into an accordion-folding packet 700 having four markable sheets 701, 702, 703 and 704. The packet 700 of FIG. 7D is also configured to securely hold inserts having at least one testing instrument, such as, for example, the insert 200 of FIG. 5D. In FIGS. 7D-E, the insert 200 is inserted into the packet 700 between the markable sheets 701 and 702 such that the testing instruments 209, 210, 211 and 212 are facing a face 750A of the markable sheet 701 when the packet 700 is folded up. The markable sheet 701 also has a back face 750B. The mirror 600 can also be placed into the packet 700 between markable sheets 703 and 704 such that the reflective surface of face 601 faces a front face 751A of the markable sheet 703 when the packet 700 is folded up. The mirror 600 also helps protect the monofilaments in the kit from being damaged, such as by breaking, distorting and/or deforming.

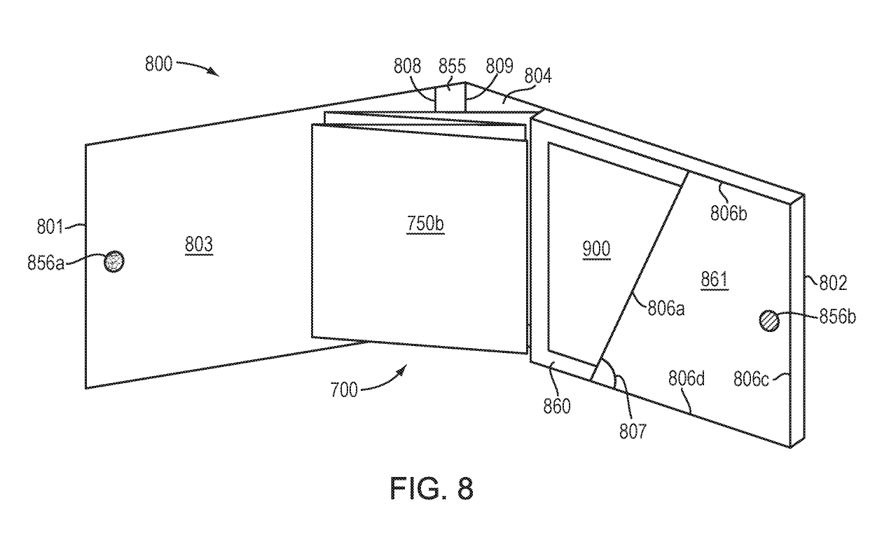

In the illustrative embodiment of FIG. 8, a testing kit for diabetes is comprised of a rectangular folder 800 made from one continuous piece of material (e.g. cardboard or the like) and includes the square packet 700 from FIGS. 7D-E, which further includes the insert 200 (not shown, and having four testing instruments, also not shown) and the mirror 600 (not shown). While the particular sizes of the testing instruments on the insert 200 may vary in other embodiments, in the particular illustrative embodiment of FIG. 8, it is contemplated that the specific dimensions of the holders 201, 202, 203 and 204 (not shown) are about 2-3 mm×4-5 mm, and more specifically, 2.5 mm×4.9 mm; and the specific lengths (as measured from the distal end to where the monofilaments attach to the holders) of the monofilaments 205, 206, 207 and 208 are 3.5-4.5 mm in length, and, and more specifically, 4.2 mm. In the illustrative embodiment of FIG. 8, the monofilaments are 10 gram monofilaments.

It is also contemplated that in the particular illustrative embodiment of FIG. 8 the specific dimensions of the insert 200 are 10-12 mm×10-12 mm, and more specifically, 11.5 mm×11.5 mm. In other embodiments, the shape of the insert 200 may vary, including, as a square or essentially square shape or rectangular or essentially rectangular shape.

The folder 800 has a front panel 801 and a back panel 802 joined by a spine 855 having a first crease 808 and a second crease 809. In this specific embodiment, the spine 855 has dimensions of 14-15 mm×0.3-1 mm, and more particularly 0.5 mm×14.5 mm. The front panel 801 has a front face 803 and a back face 862. In this specific embodiment, the front panel 801 and the back panel 802 both have the dimensions of about 19-20 mm (long sides)×14-15 mm (short sides), and more particularly 19.5 mm×14.5 mm. The rear panel 802 has a front face 804 with a rectangular first inner pocket 860 extending out from the face 804. In this illustrative embodiment, the rectangular first inner pocket 860 is formed by folding the continuous piece of material back over a portion of the rear panel 802 during manufacturing. Also in this specific embodiment, the first inner pocket 860 has the dimensions of about 14-16 mm×14-16 mm, and more particularly 15.2 mm×14.5 mm.

In FIG. 8, the first inner pocket 860 includes a slit 805 opening into the first inner pocket 860 and running at an angle 807 from one of the long sides of the first inner pocket 860 to the other side of the first inner pocket 860 to form a second inner pocket 861 of essentially a trapezoidal shape with sides 806A-D. In this specific embodiment, the side 806A is about 14.5-16 mm, the side 806B is about 7-8 mm, the side 806C is about 14-15 mm, and the side 806D is about 10-13 mm; and, more particularly, the side 806A is 15 mm, the side 806B is 7.5 mm, the side 806C is 14.5 mm, and the side 806D is 11.8 mm. Additional inserts, such as an insert 900, may be inserted securely into the second inner pocket 861. The angle 807 may vary in different embodiments from approximately 45°-90°. In this specific embodiment, the angle 807 is approximately 45°-90° and more specifically 75°.

The folder 800 also has a Velcro® securement 856A located on an outside edge (not labeled) of the front face 803 and a corresponding Velcro® securement 856B located on an outside edge (not labeled) of the second inner pocket 861. In the illustrative embodiment of FIG. 8, a center portion (not shown) of the Velcro® securement 856A is located at about 6.8-7.3 mm along an outside edge of the short edge of the front panel 801. The corresponding securement 856B is located at about 6.8-7.3 mm along the side 806C of the second inner pocket 861.

The two creases in the spines of the folders of the various embodiments discussed herein and the particular widths of the spines, such as the first crease 808 and the second crease 809 of spine 855 in FIG. 8, are specifically configured to allow a front panel to adequately fold over a rear panel which may have one or more folders, pockets and/or inserts attached to or otherwise secured therein. For example, in FIG. 8, the packet 700 includes and secures the mirror 600 (as shown in FIGS. 7A-E) and the insert 200 (having four testing instruments 209, 210, 211 and 212 glued thereon as shown, for example, in the illustrative embodiment of FIG. 7D). The packet 700 is inserted into the first inner pocket 860 and the insert 900 (e.g. instructions, etc.) is inserted into the second inner pocket 861. The front panel 801 can then be folder over the rear panel 802, the first inner pocket 860 and the second inner pocket 861, so as to effectively secure the insert 900 and the packet 700 therein (and the mirror 601 and the insert 200 also secured therein). The securements 856A and 856B can then be attached to each other to maintain the folder 800 and its contents securely in a closed position.

In the particular illustrative embodiment of FIG. 8, the specific dimensions of the packet 700 are 45-60 mm×12-13 mm when measured in an unfolded fashion (i.e. as if the markable sheets, not shown, of the packet 700 are laid flat on a surface), and more specifically, 50 mm×12.5 mm. In this specific embodiment, each of the markable sheets (not shown) of the packet 700 are 12-13 mm×12-13 mm, and more specifically, 12.5 mm×12.5 mm. Also, in this specific embodiment, the dimensions of the mirror 600 are 8-13 mm×8-13 mm, and more specifically, 11.5 mm×11.5 mm. The shape of the markable sheets and/or the mirror may be rectangular or square-like in different embodiments.

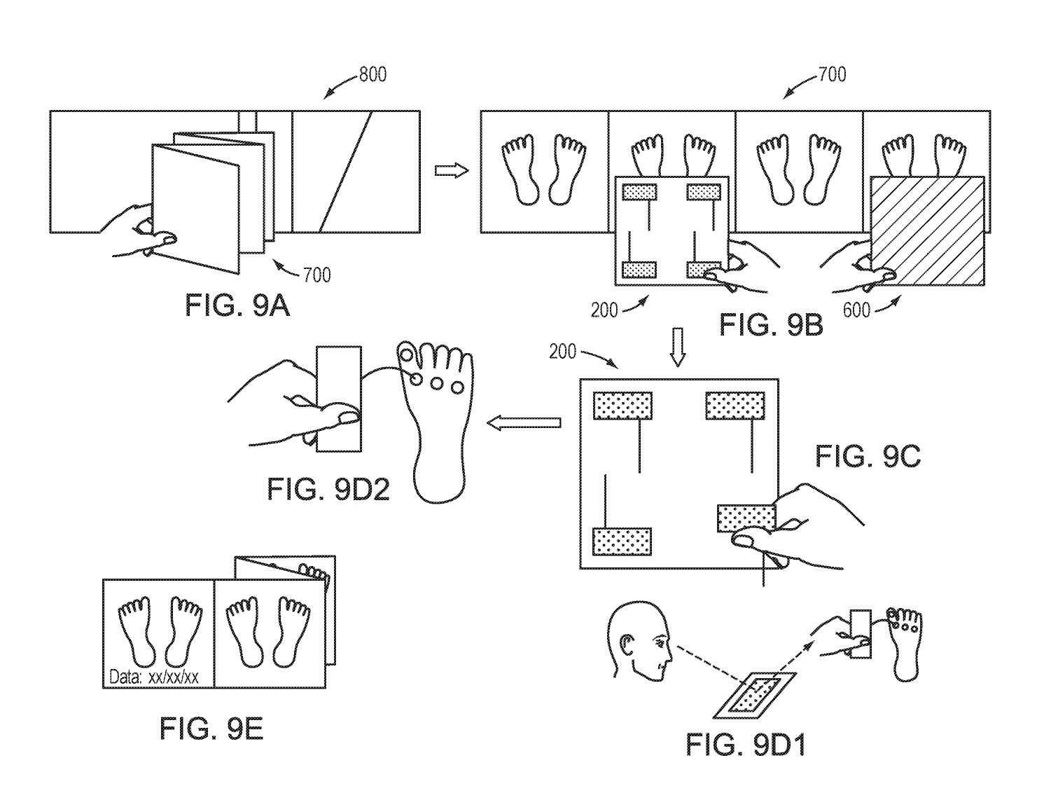

FIGS. 9A-D depict an illustrative embodiment of a method for using some of the embodiments described herein to screen for LOPS. In FIG. 9A, a user is provided with a diabetes testing kit comprising, for example, the folder 800 of FIG. 8. In FIG. 9B, the user then removes the insert 200 and the mirror 600. Next, in FIG. 9C, the user removes at least one of the one or more testing instruments that are attached to the insert 200 (e.g. by glue) by detaching a holder of the one or more instruments from the insert 200.

In FIGS. 9D1 and 9D2, the user holds the testing instrument by the holder and touches a distal end of the monofilament to one or more areas of a planter's aspect of the user's right or left foot for 1-2 seconds with enough force to bend (buckle) the monofilament. In the illustrative embodiments of FIGS. 9D1 and 9D2, the four areas of the planter's aspect of the user's right or left foot may include areas corresponding to the big toe, the first metatarsal phalangeal joint, the second/third/fourth metatarsal heads, and the fifth metatarsal head.

Optionally, a mirror, such as mirror 600, may also be used to view the user's feet while the user presses the testing instruments against the user's skin. In other embodiments, the user may also use the mirror to periodically view the user's feet (e.g. daily, weekly or monthly) to check for abnormalities such as ulcers, calluses and/or sears.

In FIG. 9E, the user then marks/records (e.g. by pen, pencil and/or sticker or the like, or any combination thereof) with a “+” or “−” on a corresponding area of the markable sheet whether the user felt the pressure of the monofilament on the at least one of four areas touched. In other embodiments, the testing instrument(s) may be touched similarly to one or more areas of skin on other areas of the user's body.

After screening for LOPS, the user may then dispose of the at least one testing instrument and reassemble the folder for delivery to the user's doctor during the next doctor's visit. Results may also be scanned, faxed, mailed or emailed to the doctor. Alternatively, the user may perform the method quarterly every three months and then subsequently reassemble the folder for delivery to the user's doctor (or send any results to the doctor by fax, mail, email, etc.). In other embodiments, a user may perform the method as frequently as needed, such as, for example, every month. Each of the folders discussed above and the contents therein may be safely and securely stored for later use or reference, etc.

The embodiments described herein overcome many of the disadvantages of current techniques and devices. For example, rather than traveling to a doctor's office to test for certain conditions, such as diabetic neuropathy, people can easily use these diabetes testing kits in the comfort of their own homes. The embodiments described herein also provide a complete, organized, relatively simple way for testing for diabetic neuropathy, including severe diabetic neuropathies, and other neuropathies. The kits provide a means to record test results, instructions and a folder in which to easily and neatly store all the related documents in one place.

The testing instruments are also securely packaged so as to decrease the risk of damaging the testing instruments, including breaking, distorting or deforming of the testing instruments, when, for example, the kits are stored and/or shipped. As such, a user can order the kits described herein and have them shipped by mail directly to the user's home without the monofilaments of the testing instruments breaking en route. Once all self-screening and/or testing is completed by the user, the results may be sent to, for example, a doctor's office or other healthcare provider. By providing a reliable, convenient and low cost self-screening apparatus for diabetic neuropathy, including severe diabetic neuropathy, that is available directly to people with diabetes, the overall prevention of diabetic foot complications becomes much more likely.

Moreover, the kits described herein provide a low-cost means for the manufacturing of testing kits, such as a diabetes testing kit; and the storing and organizing of all of the parts of a testing kit for testing for neuropathies, specifically diabetic neuropathies. For example, the diabetes testing kit depicted in the illustrative embodiment FIG. 8 comprises, among other things, the folder 800, the first inner pocket 860 and the second inner pocket 861, which are all part of, and formed from, the same continuous piece of material (e.g. one cardboard sheet). Thus, material costs and assembly costs are significantly reduced. These are some of the many advantages of the methods and apparatuses described herein.

The foregoing description has been directed to specific embodiments. It will be apparent, however, that other variations and modifications may be made to the described embodiments with the attainment of some or all of their advantages. For instance, it is expressly contemplated that the embodiments described herein may include additional components, such as additional monofilaments (e.g. two, three, four, etc. inserts with testing instruments) fliers, handouts and other material capable of being inserted into the apparatuses or used in conjunction with the methods of using the apparatuses discussed herein. It is also expressly contemplated that the embodiments described herein may include different arrangements of the testing instruments on the one or more inserts, such as, for example, reversing the directions of the testing instruments so as have the holders centrally located and the monofilaments extending outwards towards the edges of the one or more inserts onto which they are attached.

Further, while a particular order of particular methods for using the apparatuses (e.g. the kits and their constituent elements such as the testing instruments, folders, inserts, packets, etc.) have been shown and described, those skilled in the art will appreciate that other method orders, arrangements, orientations, and the like, may be used, and that the systems and methods described herein are merely illustrative embodiments. Accordingly, this description is to be taken only by way of example and not to otherwise limit the scope of the embodiments herein. Therefore, it is the object of the appended claims to cover all such variations and modifications as come within the true spirit and scope of the embodiments herein.

What is claimed is:

1. A testing kit for diabetic neuropathy comprising:
a folder with an inner pocket, the folder having an open position and a closed position;
a securement configured to secure the folder in the closed position;
an insert configured to insert into the inner pocket; and
a testing instrument attached to the insert, the testing instrument including a monofilament with a distal end and a proximal end and a holder attached to the proximal end.

2. The testing kit of claim 1 wherein two or more testing instruments are attached to the insert.

3. The testing kit of claim 1 wherein the testing instrument is attached to the insert by an adhesive.

4. The testing kit of claim 1 wherein the folder has a front panel and a rear panel joined by a spine, the rear panel having a front face and a back face and the at least one inner pocket on the front face of the rear panel.

5. The testing kit of claim 1 wherein the spine has two creases.

6. The testing kit of claim 5 wherein the inner pocket includes a first inner pocket and a second inner pocket, the second inner pocket located on a front face of the first inner pocket.

7. The testing kit of claim 1 wherein the insert has a front face and a back face and four sides, and four testing instruments are attached to each of four corners of the front face, and with the testing instruments arranged so as to have the distal ends of each of the monofilaments facing away from at least one of the four sides.

8. The testing kit of claim 1 further comprising a foldable packet with the insert secured therein, the foldable pocket configured for insertion into the inner pocket.

9. The testing kit of claim 9 further comprising a mirror secured within the foldable packet.

10. The testing kit of claim 1 wherein four or more testing instruments are attached to the insert.

11. A diabetic foot testing kit comprising:
one or more monofilaments configured to test for diabetic neuropathies of the foot;
an enclosure having a front panel and a rear panel, the enclosure adapted to secure the one or more monofilaments within the enclosure.

12. The diabetic foot testing kit of claim 12 wherein the rear panel has a front face and a back face and the front face includes at least one inner pocket covering a majority of the front face.

13. The diabetic foot testing kit of claim 12 further comprising a compact mirror.

14. A method for self-screening for severe diabetic neuropathies comprising:
providing a kit having a folder including an insert with one or more testing instruments attached thereto and a packet for recording test data, the one or more testing instruments having at least one monofilament with a distal end and a proximal end and a holder attached to the proximal end;
removing at least one of the testing instruments from the kit;
holding the holder and touching the distal end of the at least one monofilament to one or more areas of a user's skin so as to bend the at least one monofilament; and
recording on one or more markable sheets of the packet whether the user can feel the touching of the distal end of the at least one monofilament.

15. The method of claim 14 wherein the one or more areas of the user's skin is one or more areas on a plantar aspect of the user's foot and the step of touching includes touching the distal end of the at least one monofilament to the one or more areas on the plantar aspect of the user's foot.

16. The method of claim 15 wherein the one or more areas on a plantar aspect of the user's foot is four areas.

17. The method of claim 16 wherein the packet includes one or more visual representations of a right foot and a left foot and the step of recording includes marking locations on the one or more representations that correspond to the four areas touched by the at least one monofilament.

18. The method of claim 17 wherein the one or more monofilaments includes four monofilaments and the steps of removing, holding, touching and recording are performed on a regular schedule.

19. The method of claim 14 further comprising removing a mirror from the kit and using the mirror to view a plantar aspect of the user's feet.

20. The method of claim 19 wherein using the mirror to view the user's feet includes viewing the one or more areas of the user's feet while performing the steps of holding the holder and touching the distal end of the at least one monofilament to the one or more areas of the user's skin.
